(19) United States
(12) Patent Application Publication
(10) Pub. No.: US 2024/0174608 A1
(43) Pub. Date: May 30, 2024

(54) COMPOUNDS FOR BRAIN IMAGING

(71) Applicant: The General Hospital Corporation, Boston, MA (US)

(72) Inventors: Changning Wang, Melrose, MA (US); Yulong Xu, Boston, MA (US); Can Zhang, Boston, MA (US); Rudolph E. Tanzi, Milton, MA (US)

(21) Appl. No.: 18/279,162

(22) PCT Filed: Mar. 11, 2022
(86) PCT No.: PCT/US2022/019910

§ 371 Date: Aug. 28, 2023

Related U.S. Application Data
(60) Provisional application No. 63/160,652, filed on Mar. 12, 2021.

(51) Int. Cl. C07D 209/88; A61K 51/04; C07B 59/00

(52) U.S. Cl. CPC C07D 209/88; A61K 51/0446; C07B 59/002; A61K 2123/00; C07B 2200/05

(57) ABSTRACT

The present application provides radiolabeled tetrahydrocarbazole compounds that can be used as positron emission tomography imaging probes. Pharmaceutical compositions and methods of using these compounds for diagnosis and monitoring treatment of neurodegenerative diseases (such as Alzheimer's disease) are also provided.

### CLAIM OF PRIORITY

This application claims priority to U.S. Patent Application Ser. No. 63/160,652, filed on Mar. 12, 2021, the entire contents of which are hereby incorporated by reference.

### FEDERALLY SPONSORED RESEARCH OR DEVELOPMENT

This invention was made with Government support under Grant No. AG015379 awarded by the National Institutes of Health. The Government has certain rights in the invention.

### TECHNICAL FIELD

This invention relates to modulators of sirtuin 1 (SIRT1) modulators, and more particularly to tetrahydrocarbazole derivatives that can be used as positron emission tomography (“PET”) imaging probes.

### BACKGROUND

There are numerous deadly diseases affecting current human population. For example, neurodegenerative diseases affect a significant segment of population, especially the elderly. Alzheimer's disease (“AD”), a neurodegenerative disorder that affects approximately 44 million people world-wide, is the sixth leading cause of death with an estimated socioeconomic burden of more than $200 billion.

### SUMMARY

Aging is an inevitable physiological process and the biggest risk factor of Alzheimer's disease. Sirtuin 1 (SIRT1) deacetylase is an enzyme involved in multiple molecular processes in the brain, and has been implicated in the pathophysiology of neurodegenerative diseases. By binding to and modulating activity of SIRT1, the radioisotope (such as ¹¹C and ¹⁸F) labeled compounds of this disclosure are capable of visualizing SIRT1 in brains related to aging and AD, for example, by positron emission tomography (PET) imaging. Hence, these imaging tracers allow to visualize aging-related neuropathological changes in the brain and serve as useful biomarkers in elucidating neuroanatomical mechanisms of neurodegenerative diseases such as AD. Compounds of this disclosure advantageously display desirable brain uptake and selectivity, as well as stable metabolism and proper kinetics and distribution in rodent and nonhuman primate (NHP) brains.

In one general aspect, the present disclosure provides a compound of Formula (I):

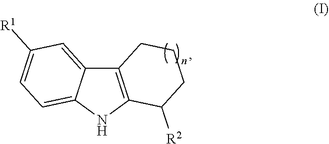

or a pharmaceutically acceptable salt thereof, wherein:
n is selected from 0, 1, and 2;
R¹ and R² are each independently selected from halo, CN, C(═O)NH₂, C₁₋₃ alkyl, C₁₋₃ haloalkyl, C₁₋₃ alkoxy, and C₁₋₃ haloalkoxy, and one of R¹ and R² comprises a radioisotope selected from ¹¹C and ¹⁸F.

In some embodiments, n is 0.

In some embodiments, n is 2.

In some embodiments, the compound of Formula (I) has formula:

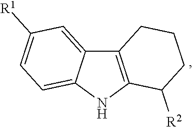

or a pharmaceutically acceptable salt thereof.

In some embodiments, R¹ comprises a radioisotope selected from ¹¹C and ¹⁸F.

In some embodiments, R¹ comprises ¹¹C.

In some embodiments, R¹ comprises ¹⁸F.

In some embodiments, R² comprises a radioisotope selected from ¹¹C and ¹⁸F.

In some embodiments, R² comprises ¹¹C.

In some embodiments, R² comprises ¹⁸F.

In some embodiments:
R¹ is selected from ¹⁸F, ¹¹CN, ¹¹C(═O)NH₂, H₃¹¹C—, F₁₈CH₂CH₂—, ¹¹CH₃O—, ¹⁸FCH₂CH₂O—, F¹⁸CH₂O—; and
R² is selected from CN and C(═O)NH₂.

In some embodiments, R¹ is H₃¹¹C.

In some embodiments, R¹ is ¹⁸F.

In some embodiments, R¹ is ¹¹CN.

In some embodiments:
R¹ is selected from halo and C₁₋₃ alkyl; and
R² is selected from ¹¹CN and ¹¹C(═O)NH₂.

In some embodiments, R² is ¹¹CN.

In some embodiments, R² is ¹¹C(═O)NH₂.

In some embodiments, the compound of Formula (I) is selected from any one of the following compounds:

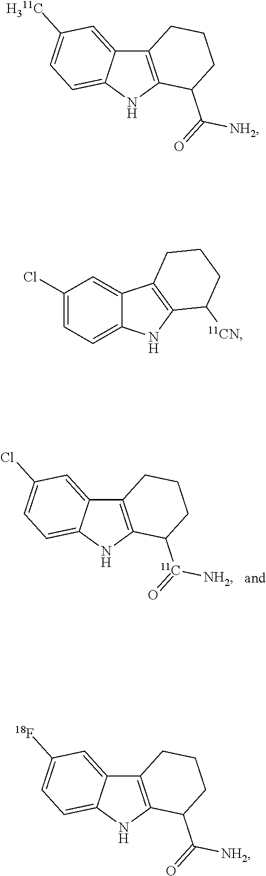

or a pharmaceutically acceptable salt thereof.

In some embodiments, the present disclosure provides a compound of formula:

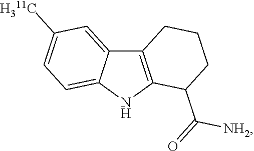 [SMILES: [11CH3]c1ccc2[nH]c3c(c2c1)CCCC3C(N)=O]

or a pharmaceutically acceptable salt thereof.

In another general aspect, the present disclosure provides a pharmaceutical composition comprising a compound of Formula (I) as described herein, or a pharmaceutically acceptable salt thereof, and a pharmaceutically acceptable carrier.

In yet another general aspect, the present disclosure provides a method of imaging a brain of a subject, the method comprising:
i) administering to the subject an effective amount of a compound of Formula (I) as described herein, or a pharmaceutically acceptable salt thereof, or a pharmaceutical composition comprising same;
ii) waiting a time sufficient to allow the compound to accumulate in the brain to be imaged; and
iii) imaging the brain with an imaging technique.

In some embodiments, the compound selectively binds to SIRT1 in the brain.

In some embodiments, imaging the brain comprises imaging midbrain, brain stem, thalamus, striatum, cerebellum, and cortex.

In some embodiments, the imaging technique is selected from positron emission tomography (PET) imaging, positron emission tomography with computer tomography (PET/CT) imaging, and positron emission tomography with magnetic resonance (PET/MRI) imaging.

In yet another general aspect, the present disclosure provides a method of monitoring treatment of a neurodegenerative disease associated with SIRT1 in a subject, the method comprising:
i) administering to the subject an effective amount of a compound of Formula (I) as described herein, or a pharmaceutically acceptable salt thereof, or a pharmaceutical composition comprising same;
ii) waiting a time sufficient to allow the compound to accumulate in a brain of the subject;
iii) imaging the brain of the subject with an imaging technique;
iv) administering to the subject a therapeutic agent in an effective amount to treat the neurodegenerative disease;
v) after iv), administering to the subject an effective amount of a compound of Formula (I) as described herein, or a pharmaceutically acceptable salt thereof, or a pharmaceutical composition comprising same;
vi) waiting a time sufficient to allow the compound to accumulate in the brain of the subject;
vii) imaging the brain of the subject with an imaging technique; and
viii) comparing the image of step iii) and the image of step vii).

In some embodiments, the imaging technique is selected from positron emission tomography (PET) imaging, positron emission tomography with computer tomography (PET/CT) imaging, and positron emission tomography with magnetic resonance (PET/MRI) imaging.

In some embodiments, the neurodegenerative disease associated with SIRT1 is selected from Alzheimer's disease (AD), amyotrophic lateral sclerosis (ALS), multiple sclerosis (MS), Parkinson's disease (PD), ischemic injury, dyskinesia, Lewy body disease, Prion disease, motor neuron disease (MND), and Huntington's disease.

In some embodiments, the neurodegenerative disease is Alzheimer's disease (AD).

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 is a chemical structure of SIRT1 modulator Ex-527 (selisistat).

FIG. 2 is a scheme showing radiosynthesis of [¹¹C]8.

FIG. 3A contains image showing representative PET sagittal images in mouse brain (time point at 15, 20, 25, 30, 35, 40, 50 and 60 min post injection). Baseline studies of [¹¹C]8 in C57BL/6 mice (25-30 g, male, n=6.

FIG. 3B contains line plot showing time-activity curves in six representative mouse brain regions. All data are the mean. Baseline studies of [¹¹C]8 in C57BL/6 mice (25-30 g, male, n=6).

FIG. 3C contains bar graph showing whole body biodistribution histogram at four different time points (5, 15, 30 and 60 min) post injection. All data are the mean±SD. The radioactivity accumulation is presented as the percentage of injected dose per gram (% ID/g). Baseline studies of [¹¹C]8 in C57BL/6 mice (25-30 g, male, n=6).

FIG. 4A contains image showing representative PET sagittal images in mouse brain (time point at 15, 20, 25, 30, 35, 40, 50 and 60 min post injection). Blocking studies with the pre-treatment of compound 8 (5 mg/kg, iv) in C57BL/6 mice (25-30 g, male, n=5).

FIG. 4B contains line plot showing time-activity curves in six representative mouse brain regions. All data are the mean Blocking studies with the pre-treatment of compound 8 (5 mg/kg, iv) in C57BL/6 mice (25-30 g, male, n=5).

FIG. 4C contains bur graph whole body biodistribution histogram at four different time points (5, 15, 30 and 60 min) post injection. All data are the mean±SD. The radioactivity accumulation is presented as the percentage of injected dose per gram (% ID/g) Blocking studies with the pre-treatment of compound 8 (5 mg/kg, iv) in C57BL/6 mice (25-30 g, male, n=5).

FIG. 5A contains image showing representative PET sagittal images in mouse brain (time point at 15, 20, 25, 30, 35, 40, 50 and 60 min post injection). Blocking studies with the pre-treatment of Ex-527 (5 mg/kg, iv) in C57BL/6 mice (25-30 g, male, n=6).

FIG. 5B contains line plot showing time-activity curves in six representative mouse brain regions. All data are the mean. Blocking studies with the pre-treatment of Ex-527 (5 mg/kg, iv) in C57BL/6 mice (25-30 g, male, n=6).

FIG. 5C contains bar graph showing whole body biodistribution histogram at four different time points (5, 15, 30 and 60 min) post injection. All data are the mean±SD. The radioactivity accumulation is presented as the percentage of injected dose per gram (% ID/g). Blocking studies with the pre-treatment of Ex-527 (5 mg/kg, iv) in C57BL/6 mice (25-30 g, male, n=6).

FIG. 6A contains image showing representative PET/MR images in the baboon brain (summed 60-90 min). PET imaging studies of [¹¹C]8 in Papio anubis baboon.

FIG. 6B contains line plot showing time-activity curves in representative baboon brain regions. PET imaging studies of [¹¹C]8 in Papio anubis baboon.

FIG. 6C contains a line plot showing arterial plasma analysis. The radioactivity accumulation is presented as the standardized uptake value (SUV). PET imaging studies of [¹¹C]8 in Papio anubis baboon.

FIG. 7A contains image showing representative PET sagittal images in wild-type mouse brain (time point at 15, 20, 25, 30, 35, 40, 50 and 60 min post injection) PET imaging studies of [¹¹C]8 in wild-type (20-25 g, male, n=4) and 5×FAD transgenic mice (20-25 g, male, n=4).

FIG. 7B contains image showing representative PET sagittal images in 5×FAD transgenic mouse brain (time point at 15, 20, 25, 30, 35, 40, 50 and 60 min post injection). PET imaging studies of [¹¹ C]8 in wild-type (20-25 g, male, n=4) and 5×FAD transgenic mice (20-25 g, male, n=4).

FIG. 7C contains line plot showing time-activity curves in the whole brain of wild-type and 5×FAD transgenic mice. All data are the mean±SD. The radioactivity accumulation is presented as the percentage of injected dose per gram (% ID/g). PET imaging studies of [¹¹C]8 in wild-type (20-25 g, male, n=4) and 5×FAD transgenic mice (20-25 g, male, n=4).

FIG. 8 is a table containing in vitro profile of Ex-527 and compound 8.

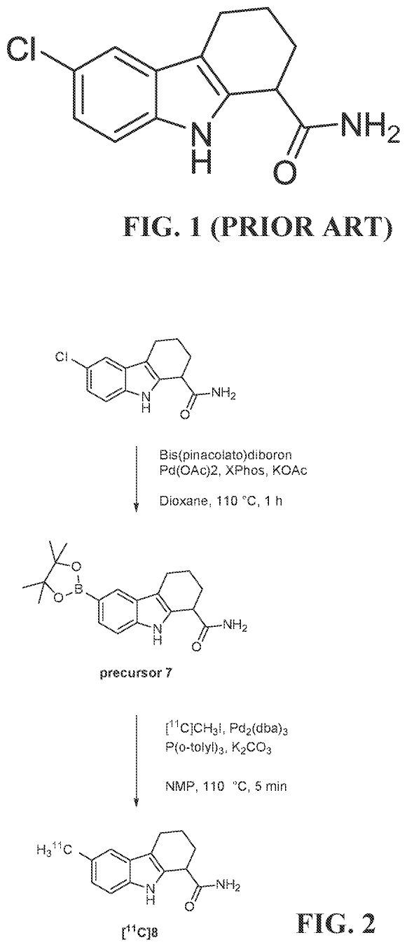

### DETAILED DESCRIPTION

### Introduction

The sirtuins (SIRTs), which refer to nicotinamide adenine dinucleotide (NAD⁺)-dependent deacetylases, are known to deacetylate N^{ϵ}-acyl-lysine of histones and other target proteins.^{1,2} SIRTs are a family of intracellular enzymes, comprising seven members, namely, SIRT1-7. These isoforms are characterized by intracellular localization: SIRT1/2 residing in nucleus or cytoplasm, SIRT3-5 residing in mitochondrion, SIRT6 residing in nucleus, SIRT7 residing in nucleolus.^{3,4} Accordingly, not only nuclear events (e.g. transcription, DNA damage repair) but also cellular events occurring in cytoplasm and mitochondrion (primarily metabolism) are regulated by SIRTs-catalyzed deacylation.^{5,6} In particular, SIRT1 can deacetylate both histones, such as H3K9, H3K14 and H4K16, and multiple non-histones, such as PPARγ, NF-Kβ and p53 tumor suppressor protein,⁷ and has been closely implicated in the pathophysiology of various human diseases, including cancer, metabolic diseases and neurodegeneration.⁸⁻¹⁰

Because of the involvement of SIRT1 in multiple molecular processes, SIRT1 has attracted considerable attention as a therapeutic target. During past few years, several chemical modulators (activators and inhibitors) for SIRT1 have been actively pursued, with the hope of developing novel therapeutic agents. However, these potential therapeutic agents have several limitations preventing their applications in imaging, such as low bioavailability, rapid metabolism, and controversial mechanism of action on SIRT1.¹²⁻¹⁴ Similarly, these compounds are poorly solubile or lack selectivity against SIRT1, making them poor imaging agents.^{15,16,17}

Disruption of the structural integrity of the SIRT1 network and interruption of SIRT1 function contribute to various age-related pathologies,¹⁸ such as Alzheimer's disease (AD). SIRT1, therefore, provides a molecular link between aging and neurodegenerative disorders. Without being bound by any theory, it is believed that SIRT1 activation may be a mechanism that closely relate to the effects of calorie restriction and NAD supplementation, both of which provide potentials in AD intervention and proven safety by oral nicotinamide.¹⁹⁻²³

Positron emission tomography (PET) imaging of SIRT1 allows to investigate SIRT1-related pathological changes between normal and disease states and in vivo interactions of SIRT1 modulators with the target. PET is a powerful noninvasive molecular imaging technique that has proven to be particularly valuable for neuronal drug discovery, as it provides quantitative information, such as target availability and target occupancy measurements, that is otherwise unattainable in the clinical setting.²⁴ Selective PET radioligands for SIRT1 of the present disclosure advantageously penetrate brain-blood barrier (BBB) and are useful for evaluating promising SIRT1 modulators for neurodegenerative diseases in clinical trials.

Despite considerable efforts to develop a suitable SIRT1 PET radioligand, hitherto no validated SIRT1 probe is available for clinical translation. Recently, a substrate-type radioligand, 2-[¹⁸F]-BzAHA, was developed for noninvasive monitoring of the spatial and temporal dynamics of SIRT1 expression-activity.²⁵ However, only PET imaging studies in rodent brain were conducted, and no further

PET imaging evaluation in living brain of nonhuman primate (NHP) was performed as a step closer toward a potential use in human, which impeded its further clinical translation. As such, the compounds of this disclosure fulfill a critical demand for SIRT1 PET radioligands with appropriate performance characteristics for SIRT1 imaging in the brain.

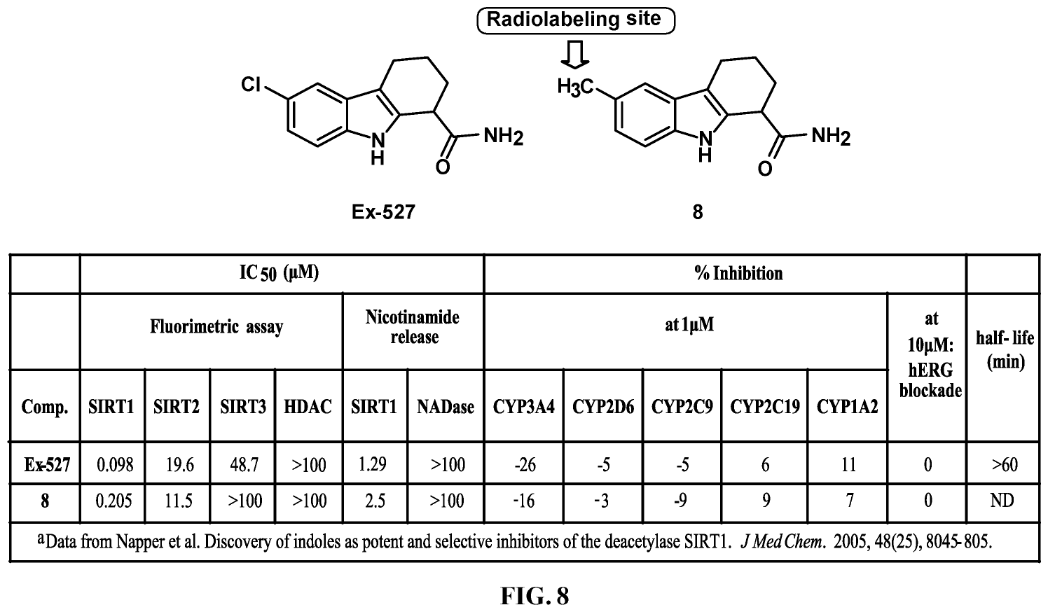

Ex-527, now named selisistat, is a highly potent and selective small molecule inhibitor against SIRT1 (IC₅₀=0.098 μM), and currently under clinical trials for the treatment of Huntington's disease.²⁶ Ex-527 exhibits much lower inhibitory activity against SIRT2 (IC₅₀=19.6 μM) and SIRT3 (IC₅₀=48.7 μM) and shows no inhibitory effect on class VII histone deacetylases (HDACs) and NAD glycohydrolase (NADase) at 100 μM (listed in FIG. 8).²⁷ The present disclosure provides compounds that are radiolabeled derivatives of Ex-527 as well as the results of in vivo imaging studies. For example, compound 8 exhibits favorable profile on SIRT1, and the 6-methyl group on compound 8 provides radiolabeling position for introducing carbon-11 or fluorine-18 isotope (e.g., 6-[¹¹C]methyl-2,3,4,9-tetrahydro-1H-carbazole-1-carboxamide ([¹¹C]8)).

As the experimental results show, derivative 8 demonstrated specific binding as well as other appropriate biochemical properties in rodent and NHP brains using PET imaging. Particularly, [¹¹C]8 displayed desirable BBB penetration, brain uptake and selectivity, as well as stable metabolism and proper kinetics and distribution. The compound was used to visualize SIRT1 in brains of AD transgenic mice, compared to nontransgenic animals, allowing to detect SIRT1 in brains of Alzheimer's model animals. Experimental results also show that SIRT1 is differentially expressed across various brain areas in rodent and NHP animal models, and that midbrain and thalamus display high SIRT1 signals in both rodents and NHP, suggesting a conserved function of SIRT1. To the best of our knowledge, this is the first research work on the distribution of SIRT1 in the preclinical animal brains. Our study has provided a useful tool to visualize AD-related SIRT1 changes in the brain, which will facilitate the drug development utilizing these pathways, and provide novel insights for substantial aging-related pathways.

The compounds of this disclosure allow visualization and recapitulation of AD-related SIRT1 changes in animal brains, supporting their promising clinical potential. Furthermore, the compounds may be used to visualize AD-related SIRT1 changes in the brain in other aging-related pathways, e.g. calorie restriction and NAD supplementation.¹⁹⁻²¹ The present results show that the compounds within the present claims are SIRT1 PET tracers and can be used, e.g., as a potential biomarker to evaluate AD progression and enhance diagnosis for AD brains.

### Radiotracer Compounds

In some embodiments, the present application provides a compound of Formula (I):

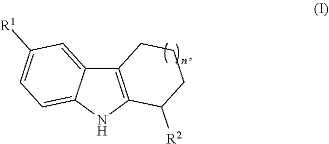

or a pharmaceutically acceptable salt thereof, wherein:
n is selected from 0, 1, and 2;
R¹ and R² are each independently selected from halo, CN, C(═O)NH₂, C₁₋₃ alkyl, C₁₋₃ haloalkyl, C₁₋₃ alkoxy, and C₁₋₃ haloalkoxy.

In some embodiments, one of le and R² comprises a radioisotope selected from ¹¹C and ¹⁸F. In some embodiments, R¹ comprises a radioisotope and R² does not comprise a radioisotope. In some embodiments, R² comprises a radioisotope and le does not comprise a radioisotope.

In some embodiments, n is 0.

In some embodiments, n is 1.

In some embodiments, n is 2.

In some embodiments, the compound of Formula (I) has formula:

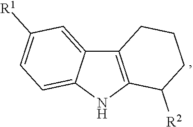

or a pharmaceutically acceptable salt thereof.

In some embodiments, R¹ comprises a radioisotope selected from ¹¹C and ¹⁸F.

In some embodiments, R¹ comprises ¹¹C.

In some embodiments, R¹ comprises ¹⁸F.

In some embodiments, R¹ is ¹⁸ F.

In some embodiments, R¹ is ¹¹CN.

In some embodiments, R¹ is ¹¹C(═O)NH₂.

In some embodiments, R¹ is C₁₋₃ alkyl comprising ¹¹C.

In some embodiments, R¹ is H₃¹¹C—.

In some embodiments, R¹ is C₁₋₃ haloalkyl comprising ¹⁸F.

In some embodiments, R¹ is selected from F¹⁸CH₂CH₂— and F¹⁸CH₂—.

In some embodiments, R¹ is C₁₋₃ alkoxy comprising ¹¹C.

In some embodiments, R¹ is ¹¹CH₃O—.

In some embodiments, R¹ is C₁₋₃ haloalkoxy comprising ¹⁸F.

In some embodiments, R¹ is selected from 18 FCH₂CH₂O— and F¹⁸CH₂O—.

In some embodiments, R¹ is selected from ¹⁸F, ¹¹CN, C₁₋₃ alkyl comprising C₁₋₃ haloalkyl comprising ¹⁸F, C₁₋₃ alkoxy comprising ¹¹C, and C₁₋₃ haloalkoxy comprising ¹⁸F.

In some embodiments, R¹ is selected from 18F, ¹¹CN, ¹¹C(═O)NH₂, H₃¹¹C—, F¹⁸CH₂CH₂—, ¹¹CH₃O—, ¹⁸FCH₂CH₂O—, and F¹⁸CH₂O—.

In some embodiments, R¹ is selected from ¹⁸F, ¹¹CN, H₃¹¹C—, F¹⁸CH₂CH₂—, ¹¹CH₃O—, ¹⁸FCH₂CH₂O—, and F¹⁸CH₂O—.

In some embodiments, R¹ is selected from ¹⁸F and H₃¹¹C—.

In some embodiments, R¹ is selected from halo, CN, C(═O)NH₂, C₁₋₃ alkyl, C₁₋₃ haloalkyl, C₁₋₃ alkoxy, and C₁₋₃ haloalkoxy.

In some embodiments, R¹ is selected from halo, CN, C₁₋₃ alkyl, and C₁₋₃ haloalkyl.

In some embodiments, R¹ is selected from halo and C₁₋₃ alkyl.

In some embodiments, R¹ is selected from Cl and CH₃.

In some embodiments, R² comprises a radioisotope selected from ¹¹C and ¹⁸F.

In some embodiments, R² comprises ¹¹C.

In some embodiments, R² comprises ¹⁸F.

In some embodiments, R² is ¹¹CN.

In some embodiments, R² is ¹¹C(═O)NH₂.

In some embodiments, R² is selected from ¹¹CN and ¹¹C(═O)NH₂.

In some embodiments, R² is selected from ¹⁸F, ¹¹CN(═O)NH₂, C₁₋₃ alkyl comprising ¹¹C, C₁₋₃ haloalkyl comprising ¹⁸F, C₁₋₃ alkoxy comprising ¹¹C, and C₁₋₃ haloalkoxy comprising ¹⁸F.

In some embodiments, R² is selected from ¹¹CN and ¹¹C(═O)NH₂.

In some embodiments, R² is selected from CN and C(═O)NH₂.

In some embodiments:
R¹ is selected from ¹⁸F, ¹¹CN, C₁₋₃ alkyl comprising ¹¹C, C₁₋₃ haloalkyl comprising ¹⁸F, C₁₋₃ alkoxy comprising ¹¹C, and C₁₋₃ haloalkoxy comprising ¹⁸F; and
R² is selected from CN and C(═O)NH₂.

In some embodiments:
R¹ is selected from halo, CN, C₁₋₃ alkyl, and C₁₋₃ haloalkyl; and
R² is selected from ¹¹CN and ¹¹C(═O)NH₂.

In some embodiments:
R¹ is selected from ¹⁸F, ¹¹CN, ¹¹C(═O)NH, H₃¹¹C—, F¹⁸CH₂CH₂—, ¹¹CH₃O—, ¹⁸FCH₂CH₂O—, and F¹⁸CH₂O—; and

R² is selected from CN and C(═O)NH₂.

In aspects of the above embodiments, R¹ is H₃¹¹C.

In aspects of the above embodiments, R¹ is ¹⁸F.

In aspects of the above embodiments, R¹ is ¹¹CN.

In some embodiments:
R¹ is selected from halo and C₁₋₃ alkyl; and
R² is selected from ¹¹CN and ¹¹C(═O)NH₂.

In aspects of the above embodiments, R² is ¹¹CN.

In aspects of the above embodiments, R² is ¹¹C(═O)NH₂.

In some embodiments, the compound of Formula (I) has formula:

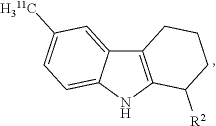

or a pharmaceutically acceptable salt thereof.

In some embodiments, the compound of Formula (I) has formula:

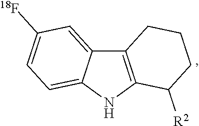

or a pharmaceutically acceptable salt thereof.

In some embodiments, the compound of Formula (I) has formula:

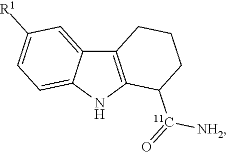

or a pharmaceutically acceptable salt thereof.

In some embodiments, the compound of Formula (I) has formula:

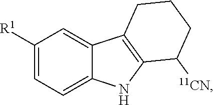

or a pharmaceutically acceptable salt thereof.

In some embodiments, the compound of Formula (I) is selected from any one of the following compounds:

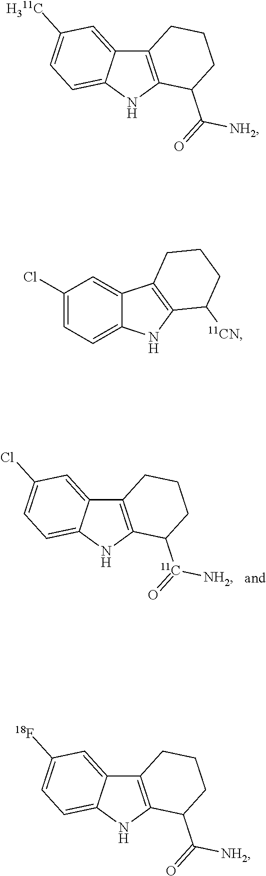

or a pharmaceutically acceptable salt thereof.

In some embodiments, the present disclosure provides a compound of formula:

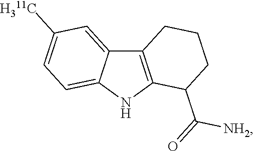 [SMILES: [11CH3]c1ccc2[nH]c3c(c2c1)CCCC3C(N)=O]

or a pharmaceutically acceptable salt thereof.

In some embodiments, the compound selectively binds to SIRT1 in the brain.

In some embodiments, any atom not designated as a radioisotope is present at its natural isotopic abundance.

### Pharmaceutically Acceptable Salts

In some embodiments, a salt of any one of the compounds of the present disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Use

In one general aspect, the present application relates to compounds of formula (I) useful in imaging techniques, diagnosing and monitoring treatment of various diseases and conditions described herein. Such compounds are labeled in so far as each compound includes at least one ¹⁸F radioisotope or at least one ¹¹C isotope.

PET has become an important clinical diagnostic and research modality, and also a valuable technology in drug discovery and development. PET offers picomolar sensitivity and is a fully translational technique that requires specific probes radiolabeled with a usually short-lived positron-emitting radionuclide. Carbon-11 (radioactive half-life (t_{1/2})=20.4 min) and fluorine-18 (t_{1/2}=109.7 min) are the most commonly used radionuclides in PET imaging. PET has provided the capability of measuring biological processes at the molecular and metabolic levels in vivo by the detection of the photons formed as a result of the annihilation of the emitted positrons.

As a noninvasive medical and molecular imaging technique and a powerful tool in neurological research, PET offers the possibility of visualizing and analyzing the target receptor expression under physiological and pathophysiological conditions. PET has often been used to detect disease-related biochemical changes before the disease-associated anatomical changes can be found using standard medical imaging modalities.

Moreover, PET tracers serve as invaluable biomarkers during the clinical development of potential therapeutics, in which the receptor occupancy of potential drug candidates in the brain is measured. In vivo receptor occupancy can help to answer many vital questions in the drug discovery and development process, such as whether potential drugs reach their molecular targets, the relationship between therapeutic dose and receptor occupancy, the correlation between receptor occupancy and plasma drug levels, and the duration of time the drug remains at its target.

Despite the great wealth of information that such probes can provide, the potential of PET strongly depends on the availability of suitable PET radiotracers. However, existing tracer discussed earlier suffer from serious drawbacks, including off-target binding, low BBB-penetration, and undesirable interaction with brain efflux pumps. The compounds within the present claims cross the BBB quickly and are mainly accumulated, e.g., in midbrain, brain stem, thalamus, striatum, cerebellum, and/or cortex, which were reported as the SIRT1-rich regions of the brain, do not engage in off-target binding, and do not interact with brain efflux pumps.

SIRT1-selective PET probes of Formula (I) are noninvasive molecular-imaging tools for quantifying spatial and temporal changes in characteristic biological markers of brain disease and for assessing potential drug efficacy. In vivo imaging of SIRT1 function in normal and pathological conditions reveals new diagnostic and therapeutic strategies for neurodegenerative disorders such as AD, which are lacking cure. Hence, the compounds of Formula (I) are useful in diagnosing a neurodegenerative disease or condition, for example, by comparing the imaged brains of healthy and ill subjects. For the treating physician, this comparison may reveal important information aiding in the diagnosis. In certain embodiments, the disease is diagnosable by imaging with SIRT1 modulator of Formula (I) because SIRT1 is implicated in the pathology of the disease.

In some embodiments, the present disclosure provides a method of identifying and/or quantifying SIRT1 density in a brain of subject. This may be attained, for example, by imaging the brain. The method includes identifying and/or quantifying SIRT1 density in midbrain, brain stem, thalamus, striatum, cerebellum, and/or cortex.

A method of imaging the brain comprises (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. Since ¹⁸F or ¹¹C within the compound of Formula (I) is a positron emitting radioisotope, the suitable imaging techniques include positron emission tomography (PET) and its modifications. As such, the imaging technique may be selected from positron emission tomography (PET) imaging, positron emission tomography with computer tomography (PET/CT) imaging, and positron emission tomography with magnetic resonance (PET/MRI) imaging, as well as other suitable methods.

In some embodiments, the present disclosure provides a method of diagnosing (or early detection) a neurodegenerative disorder (e.g., neurodegenerative disorder in which SIRT1 is implicated) in a subject, the method comprising (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. The method may also comprise comparing images obtained from subjects exhibiting the symptoms of the disease or condition with the images obtained from healthy subjects. In one example, loss or overabundance of SIRT1 receptors in the brain of the subject may be indicative of a neurodegenerative disease such as Alzheimer's disease or a related condition.

In some embodiments, the SIRT1-selective PET radiotracers of Formula (I) within the present claims are useful to study the role of SIRT1 in health and disease conditions. In some embodiments, the present disclosure provides a method of supporting a clinical development of potential therapeutics, in which the receptor occupancy of potential drug candidates such as SIRT1 modulators (inhibitors or activators) in the brain is measured. In vivo receptor occupancy can help to answer many vital questions in the drug discovery and development process such as whether potential drugs reach their molecular targets, the relationship between therapeutic dose and receptor occupancy, the correlation between receptor occupancy and plasma drug levels, and the duration of time the drug remains at its target, and similar information.

In some embodiments, the present disclosure provides a method of monitoring treatment of neurodegenerative disease (or disorder) (e.g., neurodegenerative disease in pathology of which SIRT1 is implicated) in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) to accumulate in a brain of the subject (e.g., 5 min, 15 min, or 30 min); (iii) imaging the brain of the subject with an imaging technique; (iv) administering to the subject a therapeutic agent in an effective amount to treat the neurodegenerative disorder. In one example, Aducanumab, Donepezil, Rivastigmine, Galantamine, Memantine, Suvorexant, or an experimental drug substance for treating AD may be administered to a subject undergoing treatment of AD. In another example, levodopa (L-dopa), carbidopa, safinamide, dopamine agonists (e.g., ropinirole, pramipexole, rotigotine), amantadine, trihexyphenidyl, benztropine, selegiline, rasagiline, tolcapone, entacapone, or an experimental drug substance for treating PD may be administered to a subject undergoing treatment of PD. In some embodiments, the method further includes step (v) after (iv), administering to the subject an effective amount of a compound of Formula (I), or a pharmaceutically acceptable salt thereof, or a pharmaceutical composition comprising same; (vi) waiting a time sufficient to allow the compound of Formula (I) to accumulate in the brain of the subject (e.g., 5 min, 15 min, or 30 min); (vii) imaging the brain of the subject with an imaging technique; and (viii) comparing the image of step (iii) and the image of step (vii). In one example, attaining abundance of overabundance of SIRT1 receptors in the brain of the subject, as determined by comparing the images, is indicative of successful treatment of the neurodegenerative disease (e.g., AD). Suitable examples of diseases the treatment of which can be monitored according to the methods of the present disclosure include any of the diseases described herein. One particular example is AD. Other suitable examples amyotrophic lateral sclerosis (ALS), multiple sclerosis (MS), Parkinson's disease (PD), ischemic injury, dyskinesia, Lewy body disease, Prion disease, motor neuron disease (MND), and Huntington's disease. In some embodiments, the present disclosure provides a method of monitoring treatment of Alzheimer's disease (AD), amyotrophic lateral sclerosis (ALS), multiple sclerosis (MS), Parkinson's disease (PD), ischemic injury, dyskinesia, Lewy body disease, Prion disease, motor neuron disease (MND), or Huntington's disease.

In some embodiments, the present disclosure provides a method of modulating (e.g., binding, inhibiting, or activating) SIRT1 in a cell (e.g., brain cell), the method comprising contacting the cell with an effective amount of a compound of the present disclosure (e.g., Formula (I)), or a pharmaceutically acceptable salt thereof, or a pharmaceutical composition comprising same. In some embodiments, the contacting occurs in vitro, in vivo, or ex vivo. In some embodiments, the cell is a neuron.

In some embodiments, the present disclosure provides a method of modulating (e.g., binding, inhibiting, or activating) SIRT1 in a subject, the method comprising administering to the subject an effective amount of a compound of the present disclosure (e.g., Formula (I)), or a pharmaceutically acceptable salt thereof, or a pharmaceutical composition comprising same.

Compositions, formulations, and routes of administration The present application also provides pharmaceutical compositions comprising an effective amount of a compound of the present disclosure (e.g., Formula (I)) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra- arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure (e.g., a compound of Formula (I)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0. 1 mg/kg to about 200 mg/kg; from about 0. 1 mg/kg to about 150 mg/kg; from about 0. 1 mg/kg to about 100 mg/kg; from about 0.1 mg/kg to about 50 mg/kg; from about 0. 1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, “halo” refers to F, Cl, Br, or I. In some embodiments, a halo is F, Cl, or Br.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified. Any atom identified in the compounds herein that is not specifically designated as radioisotope is present at is natural isotopic abundance.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H- 1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the RIPK1 with a compound of the invention includes the administration of a compound of the present invention to an individual or patient, such as a human, having SIRT1, as well as, for example, introducing a compound of the invention into a sample containing a cellular or purified preparation containing the SIRT1.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope.

“¹⁸F” refers to the radioisotope of fluorine having 9 protons and 9 neutrons. “¹⁸F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% 18F incorporation), at least 4500 (67.5% 18 F incorporation), at least 5000 (75% 18 F), at least 5500 (82.5% 18F incorporation), at least 6000 (90% 18F incorporation), at least 6333.3 (95% 18F incorporation), at least 6466.7 (97% 18F incorporation), at least 6600 (99% 18F incorporation), or at least 6633.3 (99.5% 18F incorporation).

“¹¹C” refers to the radioisotope of carbon having 6 protons and 5 neutrons. “C” refers to the stable isotope of carbon having 6 protons and 6 neutrons (i.e., the “¹²C isotope”). A compound of the present disclosure has an isotopic enrichment factor for each designated ¹¹C atom of at least 3500 (52.5% ¹¹C incorporation at each designated ¹¹C atom), at least 4000 (60% ¹¹C incorporation), at least 4500 (67.5% ¹¹C incorporation), at least 5000 (75% ¹¹C) at least 5500 (82.5% ¹¹C incorporation), at least 6000 (90% ¹¹C incorporation), at least 6333.3 (95% ¹¹C incorporation), at least 6466.7 (97% ¹¹C incorporation), at least 6600 (99% ¹¹C incorporation), or at least 6633.3 (99.5% ¹¹C incorporation).

### EXAMPLES

### Materials and Methods

All commercially available reagents were used without further purification unless otherwise stated. Analytical thin layer chromatography (TLC) was performed using Silica Gel GF254 plates (Merck Millipore co, .ltd, 0.2 mm thick). Compounds were purified using CombiFlash Rf 150 (Teledyne ISCO co, .ltd). ¹H and ¹³C spectra were recorded on Bruker 500 MHz. Chemical shifts in ¹H NMR spectra were reported in parts per million (ppm) on the δ scale from an internal standard of CDCl₃ (7.26 ppm). Data were reported as follows: chemical shift (6 ppm), multiplicity (s=singlet, d=doublet, t=triplet, q=quartet, m=multiplet, br=Broad), coupling constant in hertz (Hz), and integration. Chemical shifts of ¹³C NMR spectra were reported in ppm from the central peak of CDCl₃ (77.0 ppm) on the δ scale. MS data was recorded on Agilent Technologies 6310 quadrupole mass spectrometer.

All animal studies were carried out at Massachusetts General Hospital (PHS Assurance of Compliance No. A3596-01). The Subcommittee on Research Animal Care (SRAC) serves as the Institutional Animal Care and Use Committee (IACUC) for the Massachusetts General Hospital. SRAC reviewed and approved all procedures detailed in this paper.

PET/CT imaging was performed in anesthetized (isoflurane) mice to minimize discomfort. Highly trained animal technicians monitored animal safety throughout all procedures, and veterinary staff were responsible for daily care. All mice were socially housed in cages appropriate for the physical and behavioral health of the individual animal and were given unlimited access to food and water, with additional nutritional supplements provided as prescribed by the attending veterinary staff. PET/MR imaging was performed in an anesthetized (isoflurane) baboon to minimize discomfort. Highly trained animal technicians monitored animal safety throughout all procedures, and veterinary staff were responsible for daily care. Baboons are socially housed in cages appropriate for the physical and behavioral health of the individual animal and were fed thrice per diem, with additional nutritional supplements provided as prescribed by the attending veterinarian. Audio, video, and tactile enrichment was provided on a daily basis to promote psychological well-being. No NHP was euthanized to accomplish the research presented.

### Example 1

### Preparation of Precursor 7

A scheme showing chemical synthesis of precursor compound 7 is shown in FIG. 2. An oven-dried flask was charged with Ex-527 (50 mg, 0.20 mmol), Pd(OAc)₂ (0.9 mg, 0.004 mmol), XPhos (3.8 mg, 0.008 mmol), bis(pinacolato)diboron (152 mg, 0.6 mmol) and KOAc (58.9 mg, 0.6 mmol). The flask was capped with a rubber septum and then evacuated and backfilled with nitrogen gas (this sequence was carried out two times). 1,4-Dioxane (0.50 mL) was added via syringe. The reaction mixture was heated to 110° C. for 1 h. The reaction mixture was allowed to cool to room temperature. The crude product was purified via flash column chromatography on silica gel to provide the precursor 7 in a 96% yield (65 mg). ¹H NMR (500 MHz, CDCl₃) δ 8.64 (s, 1H), 8.01 (s, 1H), 7.62 (d, 1H, J=6.6 Hz), 7.30 (d, 1H, J=6.5 Hz), 3.73-3.69 (m, 1H), 2.79-2.71 (m, 2H), 2.18-2.11 (m, 2H), 1.97-1.85 (m, 2H), 1.36 (s, 12H). 13 C NMR (126 MHz, CDCl₃) δ 176.2, 138.4, 130.4, 128.5, 127.0, 126.2, 112.9, 110.6, 83.6, 41.8, 27.8, 25.0, 21.4, 20.9. MS (ESI) m/z 341.1 [M+H]⁺.

### Example 2

### Radiosynthesis of [¹¹C]8

A scheme showing chemical synthesis of radiolabeled compound 8 is shown in FIG. 2. [¹¹C]CO₂ was obtained via the ¹⁴N (p, α)¹¹C reaction on nitrogen with 2.5% oxygen, with 11 MeV protons (Siemens Eclipse cyclotron), and trapped on molecular sieves in a TRACERlab FX-MeI synthesizer (General Electric). [¹¹C]CH₄ was obtained by the reduction of [¹¹C]CO₂ in the presence of Ni/hydrogen at 350° C. and recirculated through an oven containing I₂ to produce [¹¹C]CH₃I via a radical reaction. Pd₂(dba)₃ (1.4 mg, 1.5 μmol), (o-Tol)₃P (5.5 mg, 18 μmol), and K₂CO₃ (2.1 mg, 15 μmol) were placed in a 3 mL dry conical vial, and then the vial was sealed and purged with nitrogen gas for 15 min. A solution of the precursor 7 (1.0 mg, 2.9 μmol) in NMP (300 μL) was added to the vial and then [¹¹C]CH₃I was bubbled through the solution by a stream of helium at room temperature. When the maximum delivery of [¹¹C]CH₃I was reached, the vial was heated at 110° C. for 5 min. The radioactive mixture containing [¹¹C]8 was then quenched by addition of an HPLC mobile phase (0.5 mL) and then applied to a reverse phase semipreparative HPLC (Phenomenex Gemini-NX 5u C18 110A, 250×10 mm, 5.0 mL/min, 55% H₂O+0.1% TFA/45% CH₃CN). A radioactive fraction having a retention time of 10.5 min was collected in a flask, and diluted in water (30 mL). The final product was reformulated by loading onto a solid-phase exchange (SPE) C-18 cartridge (Waters WAT020515 Sep-Pak Plus Short C18), rinsing with water (4×5 mL), eluting with EtOH (1.0 mL), and diluting with saline (9.0 mL). The chemical and radiochemical purity of [¹¹C]8 was tested by analytical HPLC (VARIAN Puruit XRs 5 C18, 150×4.6 mm), eluting with a gradient of 10-90% solvent B in solvent A (solvent A was 0.1% TFA in H₂O, and solvent B was CH₃CN), at a flow rate of 1.5 mL/min. Confirmation of the identity of [¹¹C]8 was achieved by co-injection with compound 8 as reference standard. For the determination of specific activity, mass (μmol) of [¹¹C]8 with a known radioactivity was determined by HPLC comparison of UV absorbance at 254 nm with those of known concentrations of non-radioactive compound 8.

### Example 3

### Radiosynthesis of [¹¹C]6

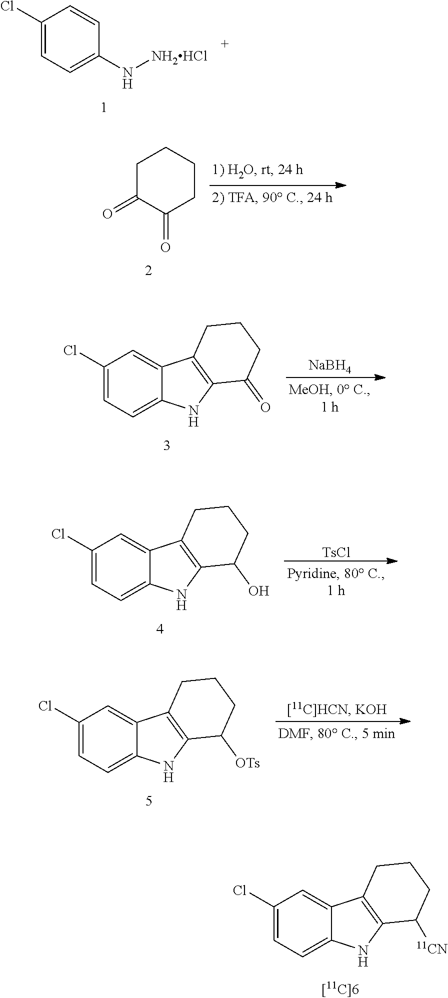

The carbazol-1-one 3 was synthesized via Fischer-indole synthesis from phenylhydrazine hydrochloride 1 and 1,2-cyclohexanedione 2. Then, the compound 3 was reduced with NaBH₄ in MeOH to afford the alcohol intermediate 4, which was coupled with tosyl chloride to provide the radiolabeling precursor 5. Precursor 5 was heated with [¹¹C]HCN in the presence of potassium hydroxide as base to yield [¹¹C]6.

### Example 4

### Radiosynthesis of [¹¹C]Ex-527

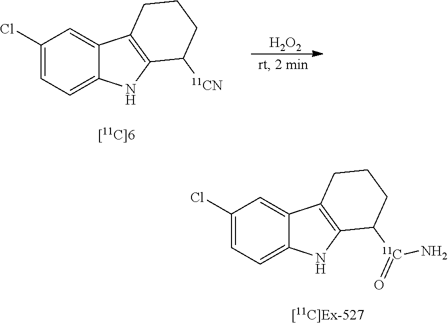

Compound [¹¹C]6 was hydrolyzed with hydrogen peroxide at ambient temperature to produce [¹¹C]Ex-527.

### Example 5

### Rodent PET/CT Imaging Studies

C57BL/6 mice were used for baseline and blocking studies (25-30 g, male; n=6 for baseline, n=5 for blocking with the pre-treatment of compound 8, and n=6 for blocking with the pre-treatment of Ex-527), and B6SJL wild-type and 5×FAD transgenic mice (20-25 g, male; n=4 for wild-type mice and n=4 for 5×FAD transgenic mice) were included to evaluate the [¹¹C]8 in a model of AD. All animals were anesthetized with inhalational isoflurane at 3% in a carrier of 2 L/min medical oxygen, and maintained at 2% isoflurane for the duration of the scan. The mice were fixed on the bed of a Triumph Trimodality PET/CT scanner (Gamma Medica, Northridge, CA) in the prone position, and injected with [¹¹C]8 (150-200 μL, ˜200 μCi) via a lateral tail vein catheterization at the start of PET acquisition. For blocking studies, compound 8 (5 mg/kg, iv) or Ex-527 (5 mg/kg, iv) was injected at 10 min prior to [¹¹C]8 injection. Dynamic PET acquisition lasted for 60 min and was followed by CT for anatomic coregistration. PET data were reconstructed using a 3D-MLEM method resulting in a full width at half-maximum resolution of 1 mm. Reconstructed images were exported from the scanner in DICOM format along with an anatomic CT. These files were imported to PMOD software (PMOD Technologies LLC, version 4.0). Volumes of interests, including the whole brain, striatum, cortex, thalamus, midbrain, cerebellum and brain stem were placed referencing the MRI template software. Time-activity curves were exported in terms of decay corrected activity at specified time points with gradually increasing intervals. The time-activity curves were expressed as the percentage of injected dose per gram (% ID/g).

### Example 6

### Rodent PET/CT Imaging Studies

C57BL/6 mice were used for baseline and blocking studies (25-30 g, male; n=6 for baseline, n=5 for blocking with the pre-treatment of compound 8, and n=6 for blocking with the pre-treatment of Ex-527), and B6SJL wild-type and 5×FAD transgenic mice (20-25 g, male; n=4 for wild-type mice and n=4 for 5×FAD transgenic mice) were included to evaluate the [¹¹C]8 in a model of AD. All animals were anesthetized with inhalational isoflurane at 3% in a carrier of 2 L/min medical oxygen, and maintained at 2% isoflurane for the duration of the scan. The mice were fixed on the bed of a Triumph Trimodality PET/CT scanner (Gamma Medica, Northridge, CA) in the prone position, and injected with [¹¹C]8 (150-200 μL, ˜200 μCi) via a lateral tail vein catheterization at the start of PET acquisition. For blocking studies, compound 8 (5 mg/kg, iv) or Ex-527 (5 mg/kg, iv) was injected at 10 min prior to [¹¹C]8 injection. Dynamic PET acquisition lasted for 60 min and was followed by CT for anatomic coregistration. PET data were reconstructed using a 3D-MLEM method resulting in a full width at half-maximum resolution of 1 mm. Reconstructed images were exported from the scanner in DICOM format along with an anatomic CT. These files were imported to PMOD software (PMOD Technologies LLC, version 4.0). Volumes of interests, including the whole brain, striatum, cortex, thalamus, midbrain, cerebellum and brain stem were placed referencing the MRI template software. Time-activity curves were exported in terms of decay corrected activity at specified time points with gradually increasing intervals. The time-activity curves were expressed as the percentage of injected dose per gram (% ID/g).

### Example 7

### NHP PET/MR Imaging Studies

A Papio Anubis baboon (19.5 kg, female), deprived of food for 12 h prior to the study, was included in the PET/MR scans. Atropine 0.05 mg/kg was used intramuscularly to prevent excessive secretion (15 or 30 min of ketamine and xylazine). Anesthesia was induced with intramuscular xylazine (0.5-2.0 mg/kg) and ketamine (10 mg/kg). After endotracheal intubation, anesthesia was maintained using isoflurane (1-1.5%, 100% O₂ or 50/50 O₂/N₂O, 1 L/min). During preparation and imaging experiment, physiological parameters were monitored and recorded, including heart rate, RR, O₂ saturation, BP, and ETCO₂, every 15 min. The baboon was catheterized antecubitally for the injection of [¹¹C]8 (5.37 mCi), and a radial arterial line was placed for metabolite analysis. PET/MR images were acquired in a Biograph mMR scanner (Siemens Healthcare, Erlangen, Germany), and PET compatible 8-channel coil arrays for NHP brain imaging with a PET resolution of ˜5 mm at isocenter. Dynamic PET image acquisition was initiated followed by administration of [¹¹C]8 in a homogeneous solution of 10% ethanol and 90% isotonic saline. A high-resolution structural MRI using MEMPRAGE sequence was acquired about 30 min post-radiotracer administration for anatomical co-registration. Dynamic PET data were acquired and stored in list mode. After corrected for attenuation, scatter, and decay, the PET images were reconstructed using a 3D-OSEM method. These files were imported to PMOD software (PMOD Technologies LLC, version 4.0). Volumes of interests were placed referencing the MRI template. Time-activity curves were exported in terms of decay corrected activity at specified time points with gradually increasing intervals. The time-activity curves were expressed as SUV.

### Discussion of Examples 1-7

Precursor 7 was synthesized for radiosynthesis of tested compound [¹¹C]8 (FIG. 2). The pinacol boronate ester 7 was synthesized by coupling commercially available Ex-527 with bis(pinacolato)diboron in the presence of Pd(OAc)2 and dialkylphosphinobiphenyl ligand XPhos as catalyst. 32 Radiosynthesis of [¹¹C]8 was successfully achieved by palladium-catalyzed methylation of precursor 7 with [¹¹C]CH₃I based on Suzuki-Miyaura coupling.³³ [¹¹C]8 was produced with a radiochemical yield of 20±5% (n=10, decay corrected) in a total synthesis time of 40±5 min from end of cyclotron bombardment. Analytical HPLC demonstrated that the radiochemical purities of [¹¹C]8 were consistently greater than 98%, and the specific activities were in the range of 1-1.5 Ci/μmol at time of injection. The resultant [¹¹C]8 was formulated in sterile saline containing <10% (v/v) ethanol and demonstrated stability sufficient for subsequent in vivo investigation.

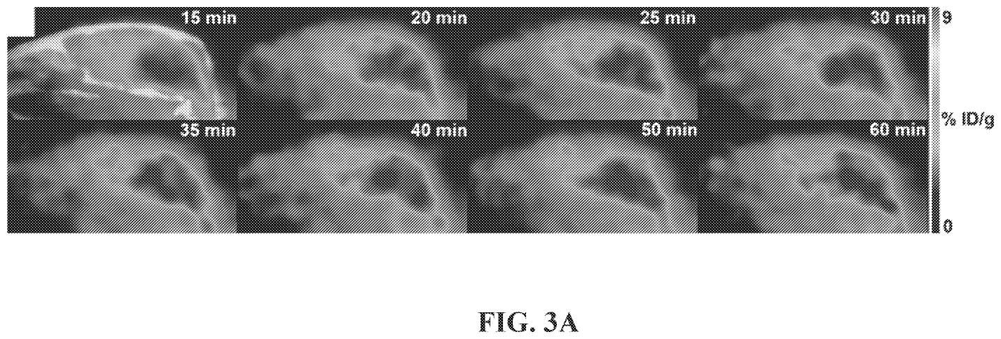

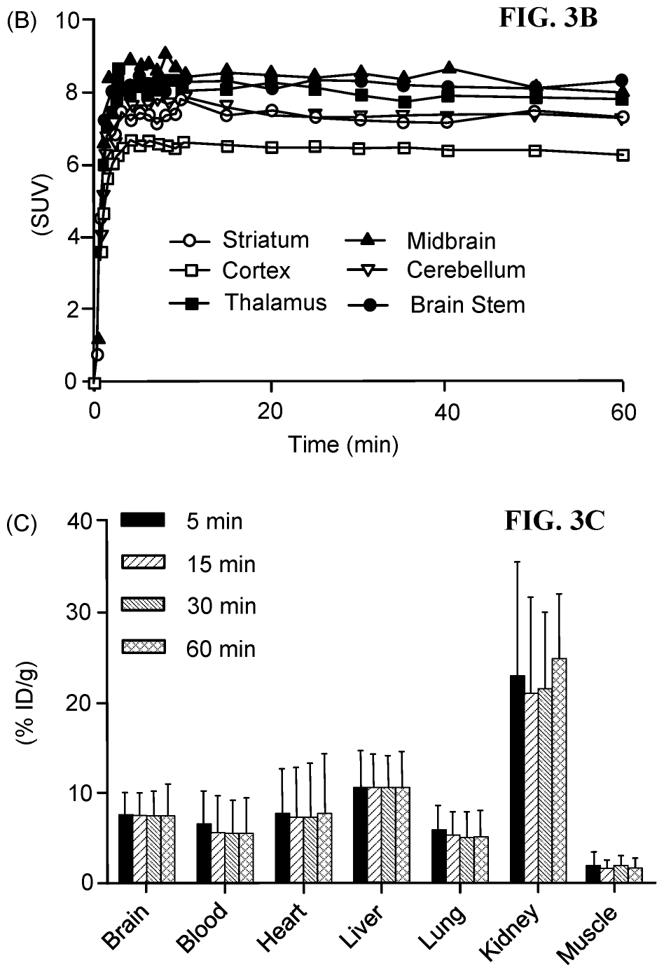

After reliable and successful radiosynthesis of [¹¹C]8, PET/CT scanning was performed to characterize its biological properties. First, biodistribution in C57BL/6 mice (25-30 g, male) was investigated. The radioactivity accumulation is presented as the percentage of injected dose per gram (% ID/g). As evident from the representative brain PET/CT images and time-activity curves in FIG. 3, [¹¹C]8 penetrated the BBB and showed excellent retention with a maximum radioactivity accumulation of 7.5% ID/g in the whole brain. The time course of [¹¹C]8 reached a plateau around 5 min. No significant washout (ratio of % ID/g_{5 min}/% ID/g_{60 min}=1.01) was observed during PET scan, indicating that indole derivative 8 might perform irreversible kinetics toward SIRT1 in brain. Additionally, [¹¹C]8 demonstrated a heterogeneous distribution with the highest brain uptake in midbrain, followed by brain stem, thalamus, striatum and cerebellum, and moderate uptake in cortex. It is worth noting that the radioactivity of [¹¹C]8 exhibited relatively low uptake in blood, followed by a slow clearance rate. The lowest level of radioactivity was observed in muscle. Other organs, including liver and kidney, exhibited high radioactivities (>10% ID/g), suggesting fast hepatobiliary and urinary elimination of [¹¹C]8.

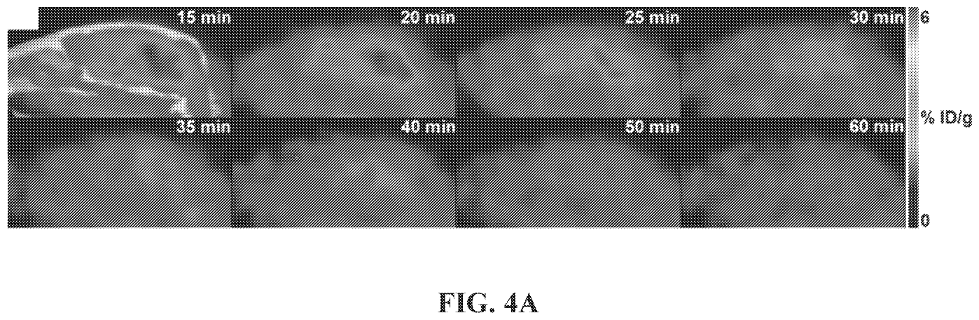

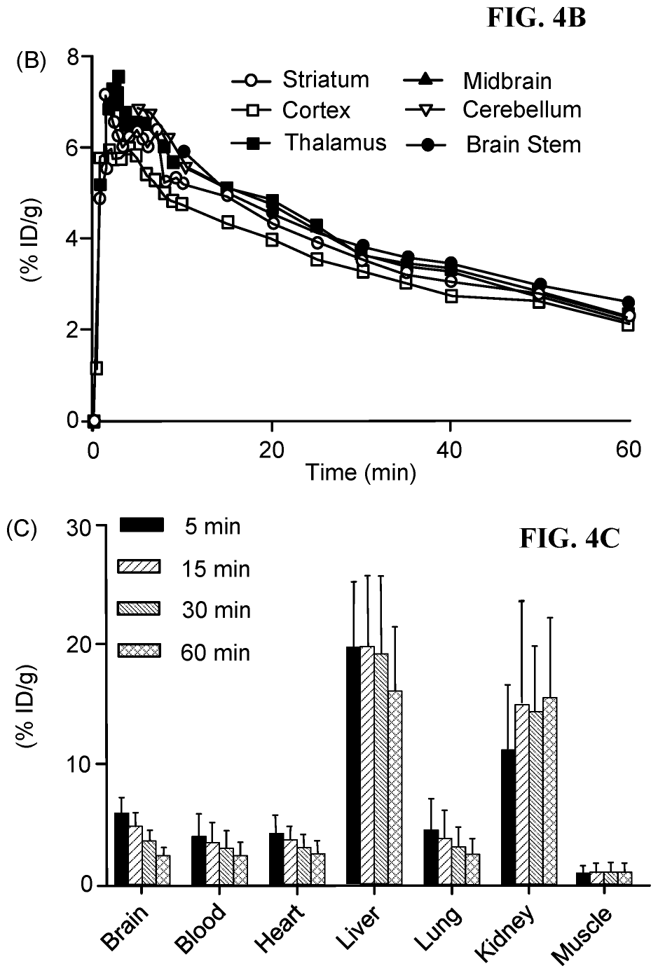

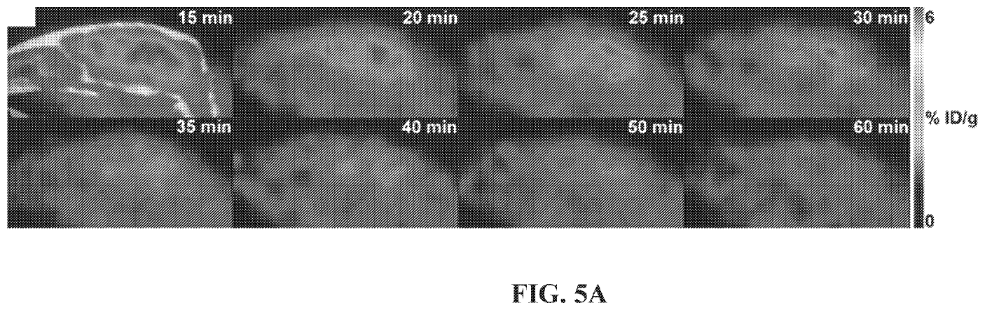

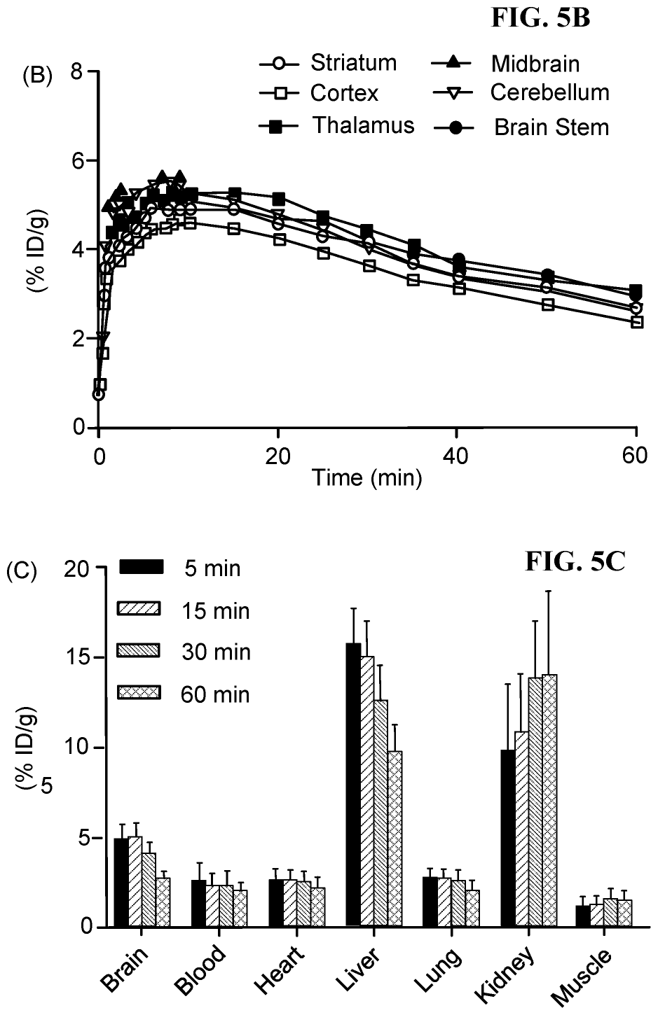

To test the in vivo specificity of [¹¹C]8 toward SIRT1, blocking experiments were carried out by the pre-treatment with compound 8 (5 mg/kg, iv) and Ex-527 (5 mg/kg, iv) respectively, 10 min prior to the radioligand administration in C57BL/6 20 mice (25-30 g, male) (FIG. 4 and FIG. 5). Time-activity curves for both blocking experiments showed remarkably decreased uptakes of [¹¹C]8 in the whole brain, followed by rapid washouts from 15 min, demonstrating excellent specific binding of [¹¹C]8 toward SIRT1. Specifically, 68% and 62% specific binding at 60 min were observed in brain at self-blocking and Ex-527-blocking experiments, respectively. As outlined in FIG. 4B (pre-treatment with compound 8), the brain uptake reached a plateau of 6.0% ID/g at 5 min, followed by a drastic decrease. However, a slow increased radioactivity during 5 to 15 min PET scan was observed in pre-treatment with Ex-527 experiments (FIG. 5B). Moreover, abundant specific binding of [¹¹C]8 was also observed in blood, heart and lung in both blocking experiments. No specific binding was found in liver and kidney, which might be related to metabolism and excretion of [¹¹C]8 and its metabolites.

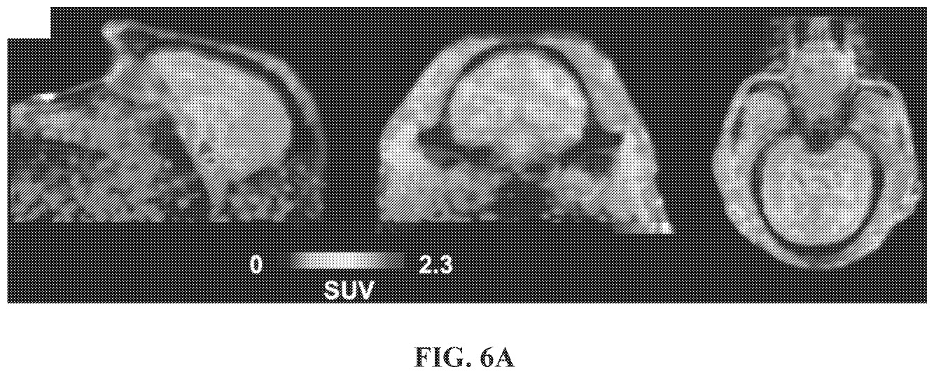

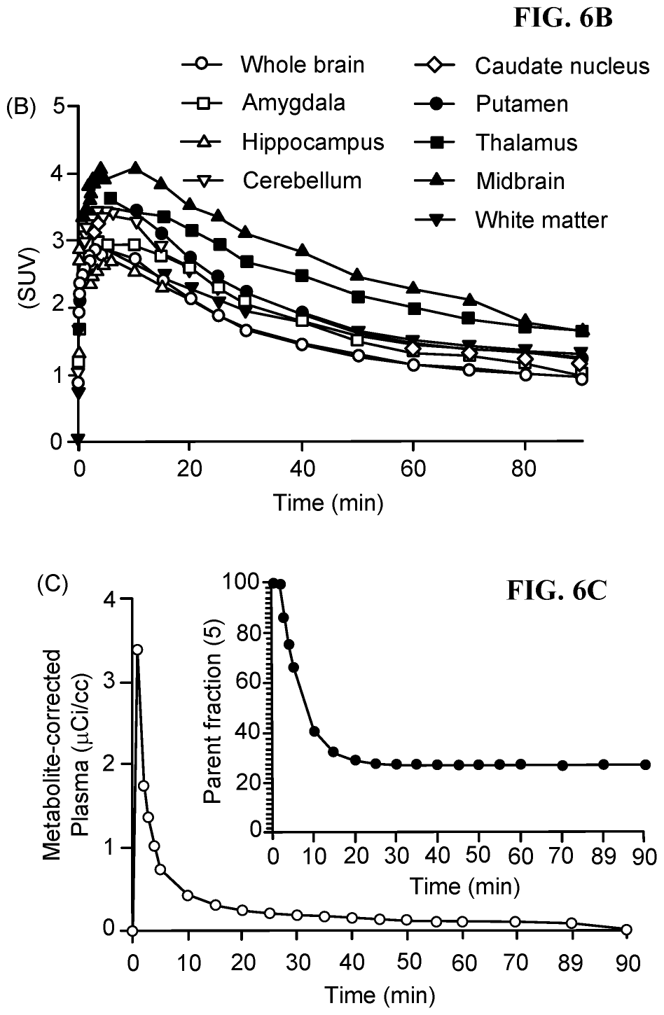

With high SIRT1-specific binding clearly demonstrated in rodent brain, [¹¹C]8 was advanced to NHP PET scanning for further assessment. Brain PET images were dynamically acquired for 90 min after intravenous administration of [¹¹C]8 to a Papio anubis baboon. As shown in FIG. 6, the whole brain PET images are co-registered to the magnetic resonance (MR) imaging images. The radioactivity accumulation is presented as the standardized uptake value (SUV). The PET/MR imaging demonstrated rapid and high brain uptake with peak whole brain uptake greater than 2.9 SUV. The time-activity curve of the whole brain indicated that [¹¹C]8 reached the peak concentration approximately 4 min followed by a slow washout over 90 min. The distribution of [¹¹C]8 in the brain was heterogeneous with the relatively higher uptake observed in the regions such as midbrain (4.1 SUV) and thalamus (3.6 SUV). Additionally, the arterial plasma washout was rapid with less than 10% of the total radioactivity after 10 min, which minimized the background signal in the brain, confirming reasonable in vivo metabolic properties. The stability of [^{C]}8 in plasma over time showed that there are still more than 30% of parent compounds at 30 min. Collectively, in both the rodent and NHP animal models, SIRT1 is differentially expressed across various brain areas. Interestingly, midbrain and thalamus display high SIRT1 signals in both rodents and NHP, suggesting a conserved function of SIRT1.

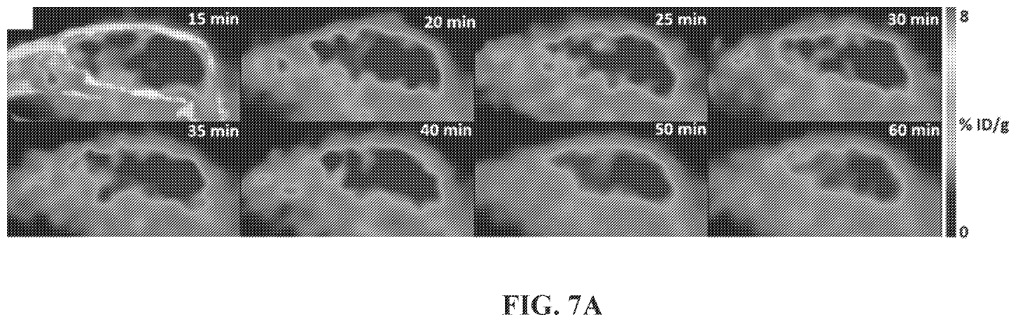

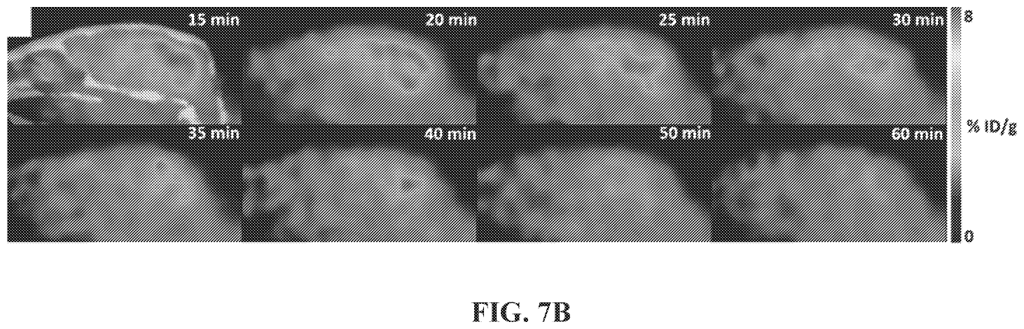

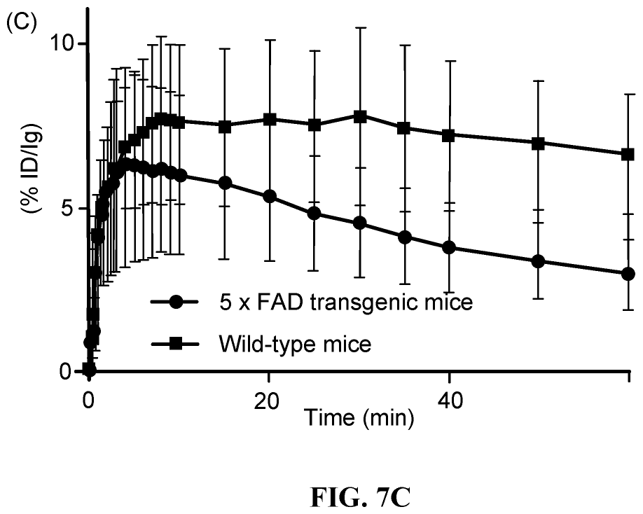

The results show that the new [¹¹C]8 PET tracer displayed desirable BBB penetration, brain uptake and selectivity, as well as stable metabolism and proper kinetics and distribution, advantageously allowing to detect SIRT1 in brains. SIRT1 has been characterized in AD associating decreased SIRT1 expressions in the brain. 34-36 The involvement of SIRT1 in AD was conducted by [¹¹C]8 PET imaging studies in wild-type and AD transgenic mice (FIG. 7). 5 x familial Alzheimer's disease (FAD) transgenic mouse models (20-25 g, male) and wild-type mice (20-25 g, male) were employed in PET/CT imaging studies with [¹¹C]8. As outlined in FIG. 7C, the brain uptakes in 5 x FAD transgenic mice reached a plateau of 6.3% ID/g at 3 min, and the brain washout was significantly increased compared with that of wide-type mice, implying that [¹¹C]8 can differentiate AD and normal brain. Surprisingly, the shape of time-activity curve in the brain of 5×FAD transgenic mice was very similar to that in brain at self-blocking experiment (FIG. 4B). These results demonstrated that the expression levels of SIRT1 decline in AD transgenic mouse brain as compared to the controls, recapiculating and supporting previous findings.^{34,35}

In summary, the present disclosure provides radiolabeling strategy for the synthesis of, e.g., [¹¹C]8 in excellent radiochemical yield and high radiochemical purity. The pharmacokinetic profile including brain uptake, clearance, binding specificity and whole body biodistribution was examined by PET imaging in rodents and further supported by NHP imaging studies. [¹¹C]8 showed excellent in vivo specific binding toward SIRT1 across multiple brain regions by pre-treatment of compound 8 and Ex-527, and can differentiate the brains between neurodegenerative diseases and normal. Hence, [¹¹C]8 is an excellent radioligand for clinical SIRT1 PET imaging.

### REFERENCES

1. Chen B, Zang W, Wang J, et al. The chemical biology of sirtuins. Chem Soc Rev. 2015;44(15):5246-5264.

2. Choudhary C, Weinert BT, Nishida Y, Verdin E, Mann M. The growing landscape of lysine acetylation links metabolism and cell signaling. Nat Rev Mol Cell Biol. 2014;15(8):536-550.

3. Folmer F, Orlikova B, Schnekenburger M, Dicato M, Diederich M. Naturally occurring regulators of histone acetylation/deacetylation. Curr Nutr Food Sci. 2010;6(1):78-99.

4. Seidel C, Schnekenburger M, Dicato M, Diederich M. Histone deacetylase modulators provided by mother nature. Genes Nutr. 2012;7(3):357-367.

5. Choi JE, Mostoslaysky R. Sirtuins, metabolism, and DNA repair. Curr Opin Genet Dev. 2014;26:24-32.

6. Sebastián C, Mostoslaysky R. The role of mammalian sirtuins in cancer metabolism. Semin Cell Dev Biol. 2015;43:33-42.

7. Bheda P, Jing H, Wolberger C, Lin H. The substrate specificity of sirtuins. Annu Rev Biochem. 2016;85:405-429.

8. Hu J, Jing H, Lin H. Sirtuin inhibitors as anticancer agents. Future Med Chem. 2014;6(8):945-966.

9. Imai S, Guarente L. Ten years of NAD-dependent SIR2 family deacetylases: implications for metabolic diseases. Trends Pharmacol Sci. 2010;31(5):212-220.

10. Min SW, Sohn PD, Cho SH, Swanson RA, Gan L. Sirtuins in neurodegenerative diseases: an update on potential mechanisms. Front Aging Neurosci. 2013;5:53.

11. Pulla VK, Battu MB, Alvala M, Sriram D, Yogeeswari P. Can targeting SIRT-1 to treat type 2 diabetes be a good strategy? A review. Expert Opin Ther Targets. 2012;16(8):819-832.

12. Marcotte PA, Richardson PL, Guo J, et al. Fluorescence assay of SIRT protein deacetylases using an acetylated peptide substrate and a secondary trypsin reaction. Anal Biochem. 2004;332(1):90-99.

13. Pacholec M, Bleasdale JE, Chrunyk B, et al. SRT1720, SRT2183, SRT1460, and resveratrol are not direct activators of SIRT1. J Biol Chem. 2010;285(11):8340-8351.

14. Dai H, Kustigian L, Carney D, et al. SIRT1 activation by small molecules-kinetic and biophysical evidence for direct interaction of enzyme and activator. J Biol Chem. 2010;285(43):32695-32703.

15. Liu T, Liu PY, Marshall GM. The critical role of the class III histone deacetylase SIRT1 in cancer. Cancer Res. 2009;69(5):1702-1705.

16. Knight JR, Milner J. SIRT1, metabolism and cancer. Curr Opin Oncol. 2012;24(1):68-75.

17. Wang Y, He J, Liao M, et al. An overview of Sirtuins as potential therapeutic target: Structure, function and modulators. Eur J Med Chem. 2019;161:48-77.

18. Lopez-Otin C, Blasco MA, Partridge L, Serrano M, Kroemer G. The hallmarks of aging. Cell. 2013;153(6):1194-1217.

19. Qin W, Yang T, Ho L, et al. Neuronal SIRT1 activation as a novel mechanism underlying the prevention of Alzheimer disease amyloid neuropathology by calorie restriction. J Biol Chem. 2006;281(31):21745-21754.

20. Araki T, Sasaki Y, Milbrandt J. Increased nuclear NAD biosynthesis and SIRT1 activation prevent axonal degeneration. Science. 2004;305(5686):1010-1013.

21. Das A, Huang GX, Bonkowski MS, et al. Impairment of an endothelial NAD(+)-H₂S signaling network is a reversible cause of vascular aging. Cell. 2018;173(1):74-89.

22. Hou Y, Lautrup S, Cordonnier S, et al. NAD(+) supplementation normalizes key Alzheimer's features and DNA damage responses in a new AD mouse model with introduced DNA repair deficiency. Proc Natl Acad Sci USA. 2018;115(8):1876-1885.

23. Chen AC, Martin AJ, Choy B, et al. A phase 3 randomized trial of nicotinamide for skin-cancer chemoprevention. N Engl J Med. 2015;373(17):1618-1626.

24. Zhang L, Butler CR, Maresca KP, et al. Identification and development of an irreversible monoacylglycerol lipase (MAGL) positron emission tomography (PET) radioligand with high specificity. J Med Chem. 2019;62(18):8532-8543.

25. Bonomi R, Popov V, Laws MT, et al. Molecular imaging of sirtuin1 expression-activity in rat brain using positron-emission tomography-magnetic-resonance imaging with [¹⁸ F]-2-fluorobenzoylaminohexanoicanilide. J Med Chem. 2018;61(16):7116-7130.

26. Arrowsmith CH, Bountra C, Fish PV, Lee K, Schapira M. Epigenetic protein families: a new frontier for drug discovery. Nat Rev Drug Discov. 2012;11(5):384-400.

27. Napper AD, Hixon J, McDonagh T, et al. Discovery of indoles as potent and selective inhibitors of the deacetylase SIRT1. J Med Chem. 2005;48(25):8045-8054.

28. Carafa V, Rotili D, Forgione M, et al. Sirtuin functions and modulation: from chemistry to the clinic. Clin Epigenetics. 2016;8:61.

29. Placzek MS, Schroeder FA, Che T, et al. Discrepancies in kappa opioid agonist binding revealed through PET imaging. ACS Chem Neurosci. 2019;10(1):384-395.

30. Fabio RD, Giovannini R, Bertani B, et al. Synthesis and SAR of substituted tetrahydrocarbazole derivatives as new NPY-1 antagonists. Bioorg Med Chem Lett. 2006;16(6):1749-1752.

31. Li X, Vince R. Conformationally restrained carbazolone-containing α,γ-diketo acids as inhibitors of HIV integrase. Bioorg Med Chem. 2006;14(9):2942-2955.

32. Billingsley KL, Barder TE, Buchwald SL. Palladium-catalyzed borylation of aryl chlorides: scope, applications, and computational studies. Angew Chem Int Ed. 2007;46(28):5359-5363.

33. Zeng F, Nye JA, Voll RJ, Howell L, Goodman MM. Synthesis and evaluation of pyridyloxypyridyl indole carboxamides as potential PET imaging agents for 5-HT_{2C} receptors. ACS Med Chem Lett. 2018;9(3):188-192.

34. Lutz MI, Milenkovic I, Regelsberger G, Kovacs GG. Distinct patterns of sirtuin expression during progression of Alzheimer's disease. NeuroMol Med. 2014;16(2):405-414.

35. Julien C, Tremblay C, Emond V, et al. SIRT1 decrease parallels the accumulation of tau in Alzheimer disease. J Neuropathol Exp Neurol. 2009;68(1):48-58.

36. Zhang Q, Liu W, Lu G. miR-200a-3p promotes (3-amyloid-induced neuronal apoptosis through down-regulation of SIRT1 in Alzheimer's disease. J Biosci. 2017;42(3):397-404.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 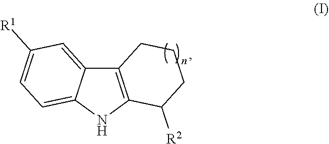
or a pharmaceutically acceptable salt thereof, wherein:
n is selected from 0, 1, and 2;
R¹ and R² are each independently selected from halo, CN, C(═O)NH₂, C₁₋₃ alkyl, C₁₋₃ haloalkyl, C₁₋₃ alkoxy, and C₁₋₃ haloalkoxy, and
one of R¹ and R² comprises a radioisotope selected from ¹¹C and ¹⁸F.

2-3. (canceled)

4. The compound of claim 1, having formula: 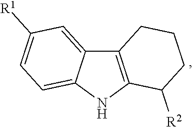
or a pharmaceutically acceptable salt thereof.

5. The compound of claim 1, wherein R¹ comprises a radioisotope selected from ¹¹C and ¹⁸F.

6. The compound of claim 5, wherein R¹ comprises ¹¹C.

7. The compound of claim 5, wherein R¹ comprises ¹⁸F.

8. The compound of claim 1, wherein R² comprises a radioisotope selected from ¹¹C and ¹⁸F.

9. The compound of claim 8, wherein R² comprises ¹¹C.

10. The compound of claim 8, wherein R² comprises ¹⁸F.

11. The compound of claim 1, wherein:
R¹ is selected from ¹⁸F, ¹¹CN, ¹¹C(═O)NH₂, H₃¹¹C—, F¹⁸CH₂CH₂—, ¹¹CH₃O—, ¹⁸FCH₂CH₂O—, and F¹⁸CH₂O—; and
R² is selected from CN and C(═O)NH₂.

12. The compound of claim 11, wherein R¹ is H₃¹¹C.

13. The compound of claim 11, wherein R¹ is ¹⁸F.

14. The compound of claim 11, wherein R¹ is ¹¹CN.

15. The compound of claim 11, wherein
R¹ is selected from halo and C₁₋₃ alkyl; and
R² is selected from ¹¹CN and ¹¹C(═O)NH₂.

16. The compound of claim 15, wherein R² is ¹¹CN.

17. The compound of claim 15, wherein R² is ¹¹C(═O)NH₂.

18. The compound of claim 1, wherein the compound of Formula (I) is selected from any one of the following compounds: 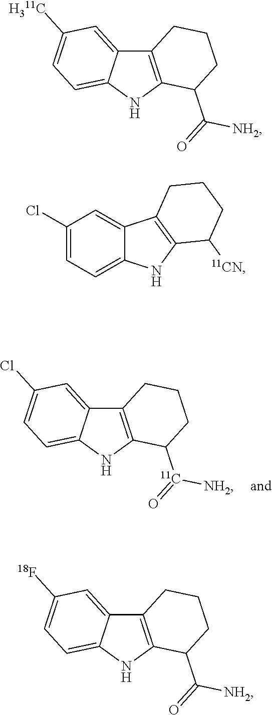
or a pharmaceutically acceptable salt thereof.

19. The compound of claim 1 having formula: 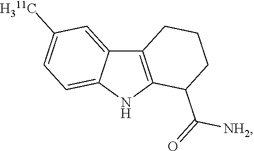 [SMILES: [11CH3]c1ccc2[nH]c3c(c2c1)CCCC3C(N)=O]
or a pharmaceutically acceptable salt thereof.

20. A pharmaceutical composition comprising a compound of claim 1, or a pharmaceutically acceptable salt thereof, and a pharmaceutically acceptable carrier.

21. A method of imaging a brain of a subject, the method comprising:
i) administering to the subject an effective amount of a compound of claim 1;
ii) waiting a time sufficient to allow the compound to accumulate in the brain to be imaged; and
iii) imaging the brain with an imaging technique.

22-24. (canceled)

25. A method of monitoring treatment of a neurodegenerative disease associated with SIRT1 in a subject, the method comprising:
i) administering to the subject an effective amount of a compound of claim 1;
ii) waiting a time sufficient to allow the compound to accumulate in a brain of the subject;
iii) imaging the brain of the subject with an imaging technique;
iv) administering to the subject a therapeutic agent in an effective amount to treat the neurodegenerative disease;
v) after iv), administering to the subject an effective amount of a compound of claim 1;
vi) waiting a time sufficient to allow the compound to accumulate in the brain of the subject;
vii) imaging the brain of the subject with an imaging technique; and
viii) comparing the image of step iii) and the image of step vii).

26-28. (canceled)
